(19) United States
(12) Patent Application Publication
(10) Pub. No.: US 2006/0147497 A1
(43) Pub. Date: Jul. 6, 2006

(54) Administration form and kit for the oral administration of active substances, vitamins and/or nutrients and use thereof

(75) Inventors: Elke Sternberger, Wuerselen (DE); Rasoul Sedaghat Kerdar, Aachen (DE); Johannes Bartholomaus, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/373,148

(22) Filed: Mar. 13, 2006

Related U.S. Application Data
(63) Continuation of application No. PCT/EP04/10145, filed on Sep. 10, 2004.

(30) Foreign Application Priority Data
Sep. 12, 2003 (DE) 103 42 513.6

(51) Int. Cl. A61K 47/00; E03B 9/20

(52) U.S. Cl. 424/439; 239/24

(57) ABSTRACT

An administration form for oral administration of active substances, vitamins and/or nutrients including a drinking straw (1) provided with a barrier device (3) which is impermeable to the active substance formulation, vitamin formulation and/or nutrient formulation (2) and which is permeable to air and to a transport liquid. The closing device has a uniform density/thickness and a hydrophobic finish.

### CROSS REFERENCE TO RELATED APPLICATIONS

This application is a continuation of international patent application no. PCT/EP2004/010145, filed Sep. 10, 2004 designating the United States of America and published in German as WO 2005/025485 on Mar. 24, 2005, the entire disclosure of which is incorporated herein by reference. Priority is claimed based on Federal Republic of Germany patent application no. DE 103 42 513.6, filed Sep. 12, 2003.

### BACKGROUND OF THE INVENTION

The invention relates to a dosage form for the oral administration of active ingredients, vitamins and/or nutrients comprising a drinking straw and a barrier device which is impermeable to an active ingredient, vitamin and/or nutrient formulation, but permeable to air and a transport liquid, wherein the barrier device exhibits a uniform density and a hydrophobic finish.

It is known to administer active ingredients, vitamins and/or nutrients with the assistance of a drinking straw which contains an active ingredient, vitamin and/or nutrient formulation which is conveyable by a liquid. Patients who have difficulty in or an aversion to taking capsules or tablets are provided in this manner with a further dosage form for active ingredients, vitamins or nutrients.

The drinking straws for the oral administration of active ingredients, vitamins and/or nutrients described in the prior art contain bungs or barrier devices which are both complicated and costly. Furthermore, known barrier devices exhibit properties which are impossible or difficult to reproduce, such that, in the event of routine investigations or repeated use in standard situations, behavior which is variable and thus unsatisfactory is obtained.

### SUMMARY OF THE INVENTION

It is therefore the object of the invention to provide a dosage form for the oral administration of active ingredients, vitamins and/or nutrients comprising a drinking straw with an active ingredient, vitamin and/or nutrient formulation, which is as simple and thus inexpensive as possible to produce and which does not exhibit the disadvantages of the dosage forms described in the prior art, as it is easier to handle and control.

The object is achieved according to the invention by the provision of a dosage form for the oral administration of active ingredients, vitamins and/or nutrients comprising a drinking straw and a barrier device which is impermeable to an active ingredient, vitamin and/or nutrient formulation, but permeable to air and a transport liquid and which exhibits a uniform density and a hydrophobic finish.

A uniform density is hereinafter understood to mean a density of the barrier device which varies by at most 10%. This is advantageous both in the case that the transport liquid moves through the barrier device when the drinking straw is sucked by the user, and in the case that the transport liquid passes by the barrier device, i.e. between the barrier device and the internal wall of the drinking straw. The hydrophobic finish ensures that as little as possible of the transport liquid remains in the barrier device, so increasing the reproducibility of the properties of the drinking straw. It is furthermore ensured in this manner that, in the event of a single (incomplete) suck, the barrier device does not become so heavy because less transport liquid remains in the barrier device. The uniform density gives rise to the advantage that the barrier device is uniformly permeable to the transport liquid over the entire cross-section of the drinking straw, so facilitating conveyance of the transport liquid. The hydrophobic finish of the barrier device and uniform density thereof combine according to the invention to ensure advantageous and in particular more highly reproducible behavior of the barrier device.

The active ingredient, vitamin and/or nutrient formulation is preferably solid or pasty, particularly preferably solid, and is hereinafter denoted according to the invention as “formulation”. A multiparticulate formulation is preferred, wherein the particles of the multiparticulate formulation preferably have a diameter of 50 μm to 1500 μm and in particular are spherical. The formulation preferably assumes the form of tablets, microtablets, granules, crystals or pellets. The formulation may also be immobilized in the drinking straw by means of a physiologically unproblematic binding material which is soluble in the transport liquid.

The active ingredients and/or vitamins and/or nutrients per se or the overall formulation may be insoluble or also soluble or at least partially soluble in the transport liquid. If the active ingredients, vitamins or nutrients or the formulations thereof are soluble in the transport liquid, they are dissolved by the transport liquid as it flows past and entrained in dissolved form. If, on the other hand, the active ingredients, vitamins or nutrients or the formulations thereof are insoluble in the transport liquid, they are suspended in the transport liquid as it flows past and entrained with the transport liquid in suspended form. Multiparticulate formulations are preferably used for this purpose. Such particles should be so small and light that they can be conveyed by the transport liquid. Adhesion of the particles to one another or to the walls of the drinking straw should be as slight as possible, which may optionally be achieved by a suitable surface treatment of the particles.

Active ingredients, preferably therapeutic active ingredients, vitamins and nutrients which may be used are any orally administrable substances in a suitable formulation. The drinking straw preferably contains a precisely determined dose of these substances, in particular a single dose for taking at one time.

The barrier device is constructed according to the invention in such a manner that it does not substantially impair the movement of the transport liquid (and also the movement of air) within the drinking straw, but instead at very most somewhat slows such movement, because it only provides a certain resistance to the passage of transport liquid (and of air) through the barrier device or past the barrier device. This is, in contrast, not the case for the formulation or the constituents thereof. The barrier device initially holds the formulation in a specific position in the drinking straw, such that it remains in the drinking straw. The barrier device must not, however, reduce the flow of the transport liquid to such an extent that a solid, insoluble active ingredient, vitamin and/or nutrient formulation can no longer be entrained. The formulation is preferably arranged in the drinking straw between the barrier device and the mouth orifice of the drinking straw, which orifice can be reached by the formulation in unimpeded manner.

The barrier device is preferably constructed as a kind of plug, the cross-section of which is preferably adapted to the cross-section of the drinking straw. This means that the cross-section of the barrier device roughly corresponds to or is optionally slightly larger than the cross-section of the drinking straw. Where each component is of a circular cross-section, the external diameter of the barrier device (when loose, i.e. not present in the drinking straw) in the preferred embodiment is thus at least as large as the internal diameter of the drinking straw. A barrier device may accordingly, when in loose form, be of a larger cross-section than the cross-section of the drinking straw. In this case, the barrier device must be compressible and be at least partially compressed before it can be introduced into the drinking straw. In order to introduce the barrier device into the drinking straw, (slight) compression of the barrier device may optionally be necessary, which ensures that the density of the barrier device is not substantially modified during this introduction operation or at least no lasting substantial variations in density over the cross-section of the barrier device are caused. In particular, a substantially constant density, i.e. a variation in density of up to at most 10%, is thus present over the entire cross-section of the barrier device.

The barrier device is preferably composed of fibers which are thermally bonded to one another. In this manner, the barrier device may be produced in a particularly simple and thus low cost manner. The fibers here in particular exhibit a thickness of 1.3 dtex to 8 dtex.

The fibers are preferably based on cellulose or cellulose derivatives and particularly preferably on cellulose acetate. The fibers may be pressed together to form a filter material so that the barrier device may preferably be produced as a plug. This plug may also consist of a corresponding nonwoven material.

The barrier device preferably corresponds to a cigarette filter. Low cost production of the barrier device is consequently possible with particularly simple means. Care must be taken to ensure that, prior to use as a barrier device, the cigarette filter is preferably hydrophobized, if the material of the cigarette filter does not already exhibit intrinsically hydrophobic properties. It is particularly preferred to use a cigarette filter as the barrier device, wherein the cigarette filter comprises cellulose acetate, in particular compacted cellulose acetate fibers.

The barrier device may preferably also be composed of a foam, preferably of an open-cell foam which is permeable to air and transport liquid. The barrier device may consequently be produced in a particularly simple manner and with particularly advantageous properties. The barrier device is particularly preferably produced from a foam of a plastics material which is approved for food use. Synthetic resin materials such as polyester, preferably polyethylene terephthalate, polystyrenes, or polyolefins, preferably polyethylene, may preferably be used to produce the barrier device.

Preferably, the fibers or the foam are/is at least partially provided with a hydrophobic finish. If the individual fibers already exhibit hydrophobic behavior, the barrier device composed of such fibers is also hydrophobic. If the individual fibers are intrinsically hydrophobic, the barrier device is denoted hereinafter as a barrier device which is also “internally” hydrophobic, such that it is completely (internally and externally) hydrophobic. The foam may similarly also be entirely hydrophobic, whether because the foamed plastics material is already intrinsically hydrophobic or because the foam has been provided with a hydrophobic finish.

The barrier device is preferably at least partially provided with an external hydrophobic finish or the barrier device comprises a peripheral zone which is more hydrophobic than the interior. Such an embodiment has the advantage that not all the fibers need individually to be provided with a hydrophobic finish, but instead only the fibers present in the outer zone of the barrier device are either intrinsically hydrophobic or are provided with a hydrophobic finish. A barrier device composed of foam may likewise be hydrophobized in the outer zone thereof. Production costs of the barrier device may be reduced by such a hydrophobic finish in only the outer zone of the barrier device. This embodiment of the barrier device is hereinafter denoted as an “externally” hydrophobic barrier device.

The hydrophobic finish of the barrier device is preferably based on polytetrafluoroethylene (Teflon™), a silicone or silicone derivative, a hydrophobic acrylic resin or another hydrophobic material, wherein a material which is approved for food use is preferably provided.

For the purposes of the present invention, a hydrophobic finish should here be taken to mean that the barrier device may be in contact with the transport liquid (for example at the lower end of the barrier device) for at least 15 minutes without the transport liquid penetrating into or through the barrier device in a quantity such that the formulation located above the barrier device is wetted. Care must be taken under this condition that the mouth orifice of the drinking straw is not sucked during the stated period of at least 15 minutes and/or that the transport liquid is not pressed from below through the barrier device during the stated period. In order to achieve this effect according to the invention—namely that the formulation (above the barrier device) is not wetted over a period of least 15 minutes despite the lower part of the barrier device being in contact with the transport liquid—it is provided in the case of a barrier device according to the invention made from foam that the foam only comprises such fine or small pores that the transport liquid as far as possible does not penetrate or at most penetrates corresponding slowly.

Acrylic resins suitable for the hydrophobic finish are preferably water-insoluble acrylic resins, such as poly(meth)acrylates, particularly preferably poly(C₁₋₄)alkyl(meth)acrylates, poly(C₁₋₄)trialkylamino(C₁₋₄)alkyl(meth)acrylates and/or the copolymers thereof, very particularly preferably copolymers of ethyl acrylate and methyl methacrylate with a molar ratio of monomers of 2:1 (Eudragit NE30D™), copolymers of ethyl acrylate, methyl methacrylate and trimethylammonium ethyl methacrylate chloride with a molar ratio of monomers of 1:2:0.1 (Eudragit RS™), copolymers of ethyl acrylate, methyl methacrylate and trimethylammonium ethyl methacrylate chloride with a molar ratio of monomers of 1:2:0.2 (Eudragit RL™) or a mixture of at least two of the stated copolymers. It is furthermore possible to use polyvinyl acetate (preferably Kollicoat SR 30 D™) or cellulose derivatives, preferably alkylcellulose, ethylcellulose or cellulose esters, for example cellulose acetate, as polymers for the hydrophobic finish. The stated polymers or copolymers may furthermore also be provided with other hydrophobic groups to increase their hydrophobic properties, such that the hydrophobic properties thereof are increased. Silicone or silicone derivatives or polytetrafluoroethylene (Teflon™) may also be used for hydrophobization.

The material used for hydrophobization according to the invention is preferably applied onto the barrier device by a spraying process or a dipping process or in general an impregnation process. It is possible in this case either that the hydrophobizing action of the hydrophobic material is achieved solely by application, i.e. directly (or after a certain reaction time) after contact with the fibers or the foam of the barrier device, or that the hydrophobizing action is obtained only after activation, for example after electromagnetic activation by means of UV radiation or other radiation or after thermal activation or also after chemical activation.

Upon application, the hydrophobic material preferably penetrates from the outside inwards into the barrier device, such that the depth of penetration into the barrier device may be influenced by the quantity of hydrophobic material applied or by the hydrophobic material selected (which in turn determines the reaction time) or optionally by the kind of activation of the hydrophobizing action. In this manner, it is possible either to use the hydrophobizing material only to finish the external zone of the barrier device or alternatively to hydrophobise the barrier device completely (internally and externally).

It is also possible to hydrophobize only the surface, and not the core, of the barrier device by means of a co-extrusion process. In this case, a for example intrinsically hydrophobic material is preferably used on the surface of the barrier device and an intrinsically non-hydrophobic material is used in the interior of the barrier device. In any event, the barrier device preferably contains only material which is unproblematic with regard to foodstuffs legislation, i.e. is approved. Low cost production of the barrier device is also made possible in this way.

The barrier device is preferably arranged movably in the drinking straw. In this way, in the event of sucking by a user, both the barrier device and the transport liquid and formulation may be moved in the same direction within the drinking straw. The position of the barrier device may thus also serve as a measure of the quantity of the formulation supplied to the user. For this reason, it is thus preferred for at least that part of the drinking straw containing the formulation or that section of the drinking straw in which the barrier device can move to be at least partially transparent and/or colored, in particular colored in such a manner that the position of the barrier device is visible from the outside. When a transparent drinking straw is used, intake of the formulation can be observed, while the use of an opaquely colored drinking straw makes it possible to conceal administration of the formulation. According to the invention, an embodiment is also possible in which the drinking straw is opaque at the beginning of the movement zone of the barrier device and is transparent at the end of said movement zone. In this way, it is possible for successful administration of the formulation to be indicated by the barrier device reaching the transparent zone, this being visible from the outside. The transparent and opaque zones may, of course, also be interchanged. The movable barrier device is preferably movable in the drinking straw only within a specific zone.

In an alternative embodiment of the invention, it is however also possible for the barrier device to be fixed in the drinking straw. The barrier action of the barrier device, i.e. its barrier action to the formulation and the constituents thereof and its permeability to the transport liquid (and to air) may more straightforwardly and reliably be achieved in this manner. The barrier device provided with a hydrophobic finish is preferably movable.

The drinking straw preferably comprises a first orifice and a second orifice facing towards the active ingredient, vitamin and/or nutrient formulation, wherein the drinking straw is narrowed at least at one of the orifices to a cross-section which is smaller in comparison with the cross-section of the barrier device. In this manner, it is very straightforwardly and thus cheaply possible to prevent the barrier device from coming away from or falling out of the drinking straw. According to the invention, the drinking straw is held upright at least in portions such that the first orifice is at its lower end and the second orifice at its upper end.

The drinking straw may comprise a bending zone or be foldable or bendable in order, on the one hand, more readily to be able to dip the lower end, i.e. the first orifice, into the transport liquid and, on the other hand, to enable easier sucking at the upper end, i.e. at the second orifice. To this end, the drinking straw is preferably oriented largely vertically in the zone of its first orifice and preferably at an angle of around 10° to 90° relative to vertical, preferably at an angle of around 30° to 70°, in the zone of its second orifice. An overall bending angle of around 0° to at most 90°, preferably of around 20° to around 60°, very particularly preferably of around 30° to around 45° is thus obtained in the position for use.

If the drinking straw is bendable, the barrier device is arranged above the bend in one and the same leg of the drinking straw as the formulation.

The drinking straw may be rigid or flexible, straight or angled, preferably reversibly bendable. The drinking straw preferably consists of a synthetic material. At least the optionally present zone in which the drinking straw is reversibly bendable is preferably produced from a resilient synthetic material or a synthetic material which is plastically deformable at a temperature below the softening point thereof. The structure of the zone in which the drinking straw is reversibly bendable preferably resembles an accordion.

The internal diameter of the drinking straw should preferably be at least 3 mm such that a sufficient quantity of transport liquid to convey the formulation can be sucked through the drinking straw. An internal diameter of 4-15 mm is preferred, an internal diameter of 5-10 mm being very particularly preferred. The drinking straw preferably has a round, oval, rectangular or square cross-section and the inside thereof is preferably smooth. A round cross-section is preferred.

The second orifice is preferably provided with a removable closing device which is impermeable to the formulation. In this manner, it is straightforwardly possible to prevent the formulation from falling out of the drinking straw. The closing device may preferably be closed and opened reversibly. In this manner, the formulation may also be supplied to a user in two or more, temporally successive sub-steps, wherein the formulation is safely enclosed by the reversibly closable closing device between said sub-steps.

This closing device is preferably a bung, a plug, a film, a mesh or a cap, such that closure of the drinking straw may be effected by very simple means. The closing device is preferably made from a material which is insoluble or at least only partially soluble in the transport liquid, is air-impermeable or at least only partially air-permeable and is preferably physiologically unproblematic. The internal diameter of the closing device is adapted to the external diameter of the drinking straw, such that the closing device may be placed on the drinking straw.

In an alternative embodiment of the invention, the closing device may preferably also take the form of a reversibly bent part of the drinking straw between the first orifice and the second orifice, which part may preferably be reversibly fixed in the bent position with the assistance of a clip, a cuff or a cap. This closes the drinking straw, i.e. the bend provided in this manner effects closure of the drinking straw at least at the second orifice thereof. A bending angle for the closed position of preferably approaching 180° is obtained in this manner.

The second orifice of the drinking straw is preferably constructed as a mouthpiece and in particular comprises ribbing. In this way, it is possible according to the invention for the formulation reliably and simply to be given even to those users of the drinking straw who, due to their age or their complaint have difficulties with swallowing or also difficulties with coordination (for example with moving their mouth).

The first orifice is preferably constructed as a connection piece to a reservoir of the transport liquid. In this way, it is particularly straightforwardly possible to provide a secure connection of the drinking straw with a reservoir of the transport liquid and so in particular to prevent spillage of the transport liquid.

The drinking straw preferably further comprises marking to indicate the mouth part and/or to indicate the direction of sucking for the transport liquid. This considerably simplifies use and handling of the drinking straw because such use is intuitively possible. It is particularly advantageous if the marking takes the form of a symbol, for example an arrow or the like. In this case, the user does not need to recognise any lettering, so making use of the dosage form according to the invention overall simpler and thus more reliable.

The present invention also provides a kit comprising the dosage form according to the invention and a physiologically acceptable transport liquid which optionally contains active ingredients, nutrients and/or vitamins and is preferably present in a container.

An aqueous liquid is preferably suitable as transport liquid, wherein water, lemonade, fruit juice without fruit pulp, tea, coffee, milk and/or a liquid comprising milk constituents are particularly preferred.

The present invention also provides the use of a cigarette filter as a barrier device for a dosage form according to the invention.

### BRIEF DESCRIPTION OF THE DRAWINGS

The invention will be described in further detail hereinafter with reference to illustrative preferred embodiments shown in the accompanying drawings, which are merely examples of dosage forms according to the invention, but are note intended to limit the scope of the invention. In the drawings:

FIG. 1 shows a dosage form according to the invention with a cap in place and a barrier device in a side view with a section line II-II;

FIG. 2 shows a cross-sectional representation of the illustrated dosage form with the barrier device along the section line II-II shown in FIG. 1;

FIGS. 3 and 4 show side view representations of two embodiments of the barrier device of the invention, and

FIG. 5 shows a representation of a kit according to the invention or a situation of use of the dosage form according to the invention.

### DETAILED DESCRIPTION OF PREFERRED EMBODIMENTS

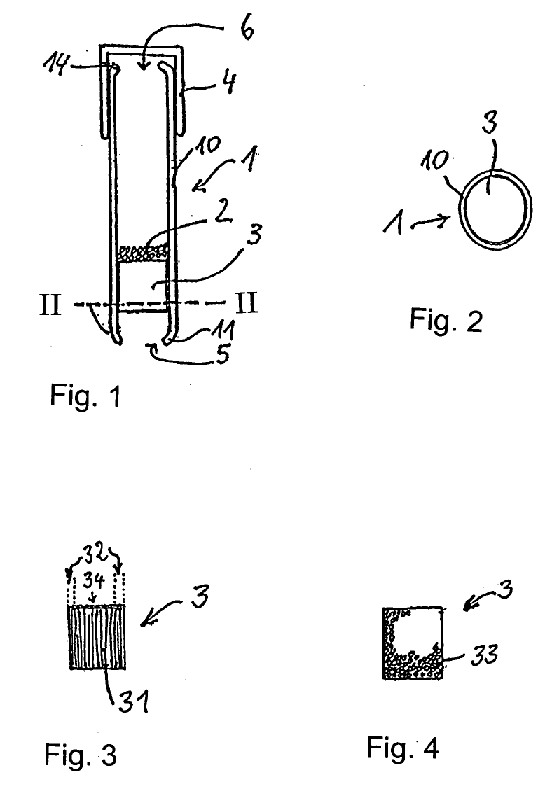

FIG. 1 shows a dosage form according to the invention which comprises a drinking straw 1 with a barrier device 3 and a formulation 2 located therein. The drinking straw 1 comprises a drinking straw wall 10, a first orifice 5 and a second orifice 6, wherein the second orifice 6 is closed by way of example with a closing device 4. The first orifice 5 is hereinafter also denoted the inlet orifice 5 and the second orifice 6 is hereinafter also denoted the mouth orifice 6. FIG. 1 furthermore shows a section line II-II across the lengthwise extension of the drinking straw 1 at the level of the barrier device 3. The formulation 2 is arranged on the side of the barrier device 3 facing towards the second orifice 6 and also remains there due to the barrier action of the barrier device 3 for the formulation 2.

The plug 3 serving as barrier device 3 consists, for example, of hydrophobic filter material, in particular in the form of a cigarette filter, and is permeable to air and a transport liquid in particular when suction is applied, but is impermeable to the formulation 2. The plug 3 is, for example, constructed as a compact, cylindrical body and is round in cross-section. The plug 3 is permanently fixed in the drinking straw 1 or alternatively arranged movably within limits. If the barrier device 3 or plug 3 is arranged movably, its mobility is in any event limited, for example already by the fact that the drinking straw 1 according to the invention preferably comprises a reduction in cross-section both at its mouth orifice 6 and at its inlet orifice 5, i.e. a reduction in size at least of the internal diameter or in general of the internal cross-section of the drinking straw 1, wherein it is preferably provided that the reduction in cross-section is also accompanied by a reduction in the external diameter of the drinking straw 1, i.e. the “wall thickness” of the drinking straw 1 is substantially constant in the zone of the reduction in cross-section. A first such reduction in cross-section is arranged by way of example in FIG. 1 at the inlet orifice 5 and a second such reduction in cross-section 14 is arranged by way of example in FIG. 1 at the mouth orifice 6. The reductions in cross-section 11, 14 ensure that the barrier device 3 (which is mobile) cannot in any event escape from the drinking straw 1 (through either orifice 5, 6). The zone between the reductions in cross-section 11, 14 could thus be denoted as the “maximum movement zone” of the barrier device 3. It may however, also be provided according to the invention that the movement zone is further limited by other narrowings in cross-section, such as a bendable zone.

FIG. 2 shows a cross-sectional representation of the drinking straw 1 along the section line II-II from FIG. 1, wherein a section through the barrier device 3 is also drawn. It may be seen here that the external diameter of the barrier device 3 is adapted to the internal diameter of the drinking straw 1 in such a manner that the formulation 2 cannot get from the side of the barrier device 3 facing the second orifice 6 to the other side. This is in particular ensured by a sufficiently small space between the wall 10 of the drinking straw 1 and the barrier device 3. This space is selected in such a manner that, if the barrier device 3 is movable, sufficient friction prevails between the barrier device 3 and the wall 10 of the drinking straw to ensure that, once it has moved into a position in the direction of the second orifice 6, the barrier device 3 may preferably no longer return to its starting position or may do so only with difficulty. The hydrophobic finish of the barrier device assists in fixing the barrier device in each position.

Persons skilled in the art will recognize that the round cross-section of both the drinking straw 1 and the barrier device 3 shown in FIG. 2 is merely selected as an example and may also be replaced by any other cross-sectional shape, for example square, rectangular, triangular, pentagonal, hexagonal or octagonal.

FIG. 3 shows a barrier device 3 composed of fibers 31 in side view. The barrier device 3 shown in FIG. 3 furthermore additionally comprises a hydrophobic peripheral zone 32. Thanks to its substantially impermeable behavior towards the transport liquid 7 or its impermeable behavior in comparison with the interior 34 of the barrier device 3, the peripheral zone 32 ensures that the transport liquid 7 substantially flows through the interior 34 of the barrier device 3 or alternatively to the side of the barrier device 3, i.e. between the barrier device 3 and the wall 10 of the drinking straw 1. The peripheral zone 32 may in particular be dispensed with if the fibers 31 of the barrier device 3 themselves assume hydrophobised form, whether because they are composed of an intrinsically hydrophobic material or alternatively because, although composed of an intrinsically less hydrophobic material, they have subsequently been hydrophobised overall. The barrier device 3 may, however, be constructed according to the invention such that it is hydrophobised both in the interior 34 and on the exterior, i.e. in the peripheral zone 32. According to the invention, the barrier device 3 may alternatively also assume the form of a hydrophobised and/or optionally hydrophobically “jacketed” cigarette filter.

FIG. 4 shows a barrier device 3 made from foam 33, in particular from open-cell foam 33, in side view. The foam 33 is in particular partially permeable and may, of course, according to the invention, such as indicated in FIG. 3 for the case of a barrier device comprising fibers, likewise be divided into a (preferably “more hydrophobic”) peripheral zone 32 and a (preferably less hydrophobic) interior 34. The pore size of the foam is selected according to the invention in such a manner that when the transport liquid comes into contact with the barrier device 3, it does not penetrate too rapidly, in particular due to the hydrophobic finish of the barrier device 3.

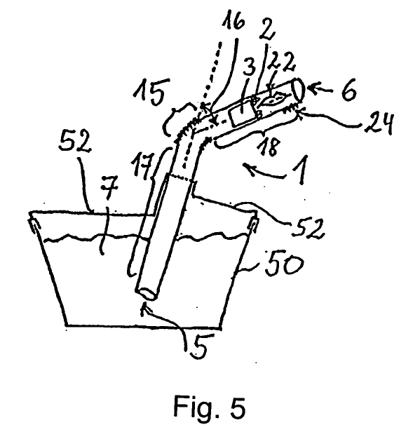

FIG. 5 shows a representation of a kit according to the invention or a situation of use of the dosage form according to the invention. The drinking straw 1 (shown purely schematically in FIG. 5) is immersed with its first orifice 5 in the transport liquid 7, which is located in a reservoir 50. By means of a suction movement by a user (not shown) on the drinking straw 1 at the second orifice 6, transport liquid 7 is sucked through the drinking straw 1 and conveys the formulation 2 or the constituents thereof to the orifice 6 and so into the user's mouth. The drinking straw 1 may here be provided so as to be foldable or bendable by a bending angle 16 in a bending zone 15. The bending zone 15 divides the drinking straw 1 into a first (lower) part 17 and a second (upper) part 18. The first part 17 is also denoted the zone 17 of the first orifice 5 and the second part 18 is also denoted the zone 18 of the second orifice 6. The zone 17 of the first orifice 5 is here oriented largely vertically and the zone 18 of the second orifice 6 is bent by the bending angle 16. The second part 18 should here be oriented at most horizontally because the liquid may otherwise flow out of the second orifice 6 in uncontrolled manner.

Where a bending zone 15 is present, the barrier device 3, like the formulation, is provided according to the invention in the upper part 18 of the drinking straw 1.

FIG. 5 furthermore schematically shows a connection piece 52 between the drinking straw and the reservoir 50, which prevents spillage of the transport liquid 7 either from the reservoir 50 or alternatively, for example due to the user ceasing to suck on the drinking straw 1, from the drinking straw 1.

FIG. 5 furthermore schematically shows a marking 22 in the form of a symbol representing a mouth on the drinking straw 1 which is located clearly closer to the second orifice 6 than to the first orifice 5, in order to indicate that, if it is to function properly, the dosage form must be sucked at the second orifice 6.

FIG. 5 furthermore shows ribbing 24 of the mouthpiece adjacent the second orifice 6 of the drinking straw 1. The purpose of the ribbing 24 is to enable the user to be able to grip the drinking straw 1 better.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A dosage form for oral administration of an active ingredient, vitamin or nutrient formulation comprising a drinking straw with a mouth orifice at a top end thereof, a charge of active ingredient, vitamin or nutrient formulation in said straw, and a barrier device which is impermeable to the active ingredient, vitamin and/or nutrient formulation but permeable to air and to a transport liquid; said barrier device being disposed below said charge and having a uniform density and a hydrophobic finish; and said hydrophobic finish being such that the barrier device may be in contact with the transport liquid for a period of at least 15 minutes without the transport liquid penetrating into or through the barrier device in a sufficient quantity to wet the formulation located above the barrier device, without application of suction to the mouth orifice or pressure from below to the transport liquid during the period of contact.

2. A dosage form according to claim 1, wherein the barrier device is composed of fibers.

3. A dosage form according to claim 2, wherein the fibers are cellulosic fibers.

4. A dosage form according to claim 3, wherein the fibers are cellulose acetate fibers.

5. A dosage form according to claim 1, wherein the barrier device is made of cigarette filter material.

6. A dosage form according to claim 1, wherein the barrier device is comprised of an open-cell, permeable foam material.

7. A dosage form according to claim 1, wherein the barrier device is made of fibers or foam material which are at least partially hydrophobic.

8. A dosage form according to claim 1, wherein the barrier device comprises an internal core surrounded by a peripheral zone, and the peripheral zone is more hydrophobic than the internal core.

9. A dosage form according to claim 1, wherein the hydrophobic finish is composed of a food-safe material selected from the group consisting of polytetrafluoroethylene, silicones and silicone derivatives.

10. A dosage form according to claim 1, wherein the barrier device is movably arranged in the drinking straw.

11. A dosage form according to claim 1, wherein at least the part of the drinking straw containing said charge is transparent or colored.

12. A dosage form according to claim 1, wherein at least one of the mouth orifice and a lower inlet orifice is narrowed to a cross-section which is smaller than the barrier device cross section.

13. A dosage form according to claim 1, further comprising at least one closing device for closing an orifice at an end of the drinking straw.

14. A dosage form according to claim 13, wherein said closing device is selected from the group consisting of a bung, a plug, a film, a mesh and a cap.

15. A dosage form according to claim 1, wherein the mouth orifice is constructed as a ribbed mouthpiece.

16. A dosage form according to claim 1, wherein said drinking straw comprises a connecting piece for connecting an inlet orifice of the drinking straw to a reservoir of a transport liquid.

17. A dosage form according to claim 1, further comprising a perceptible marking for indicating the mouth orifice or the direction of sucking of the transport liquid.

18. A method of retaining a charge of an active substance, vitamin or nutrient formulation in a drinking straw, said method comprising inserting a cigarette filter in said drinking straw underneath said charge, said cigarette filter being impermeable to the active substance, vitamin or nutrient formulation but permeable to air and to a transport liquid and having a uniform density and a hydrophobic finish, said hydrophobic finish being such that a lower end of the cigarette filter may be in contact with the transport liquid for a period of at least 15 minutes without the transport liquid penetrating through the cigarette filter in sufficient quantity to wet the formulation above the cigarette filter, without application of suction to a mouth orifice of the drinking straw or of pressure to the transport liquid underneath the cigarette filter during the contact period.

19. A kit comprising a dosage form according to claim 1, and a reservoir containing a supply of a physiologically acceptable transport liquid.

20. A kit according to claim 19, wherein the transport liquid is an aqueous liquid selected from the group consisting of water, lemonade, pulp-free fruit juice, tea, coffee, milk and liquids comprising milk constituents.
